(19) United States
(12) Patent Application Publication
(10) Pub. No.: US 2012/0180802 A1
(43) Pub. Date: Jul. 19, 2012

(54) SHAPE MEMORY DEVICES AND THEIR USE IN CONTROLLING DEVICE-ENVIRONMENT INTERACTIONS

(75) Inventors: Taylor Ware, Dallas, TX (US); Walter Voit, Dallas, TX (US)

(73) Assignee: The Board of Regents of the University of Texas System, Austin, TX (US)

(21) Appl. No.: 13/163,613

(22) Filed: Jun. 17, 2011

Related U.S. Application Data
(60) Provisional application No. 61/355,645, filed on Jun. 17, 2010.

(51) Int. Cl. A61B 17/00; B29C 67/24; H01L 41/22; B29D 11/00; B29C 69/00; C08L 47/00; H01B 13/00

(52) U.S. Cl. 128/898; 525/219; 264/129; 264/104; 264/1.1

(57) ABSTRACT

The invention is directed to shape memory polymer compositions, articles of manufacture thereof, and methods of preparation and use thereof. The invention is further directed to methods of controlling the nature of the interaction of a shape memory device with the environment in which it is operating.

### CROSS-REFERENCES TO RELATED APPLICATIONS

This Application claims priority from U.S. Provisional Patent Application No. 61/355,645 filed Jun. 17, 2010, which is hereby incorporated by reference as if fully set forth herein.

### FIELD OF THE INVENTION

The claimed invention relates to shape memory devices, methods of producing same and the use of these materials as well as methods of controlling the nature of the interaction of a shape memory device with the environment in which it is operating.

### BACKGROUND OF THE INVENTION

Shape memory is the ability of a material to remember its original shape, either after mechanical deformation, which is a one-way effect, or by cooling and heating, which is a two-way effect. This phenomenon is based on a structural phase transformation.

Materials known to have these properties are shape memory alloys (SMAs). The structure phase transformation of these materials is known as a martensitic transformation. These materials have been proposed for various applications such as vascular stents, medical guidewires, orthodontic wires, vibration dampers, pipe couplings. However, these materials have not been widely used, in part due to their relatively high costs and their limited range of mechanical properties.

Shape memory polymers (SMPs) have been under active development as a replacement or augmentation to SMAs. SMPs enjoy many advantages, among which are low density, high recoverable strain (up to several hundred percent compared to less than 8% for SMA), tailorability of the transition temperature and rubbery modulus according to the application, easy processability, and economy of materials and manufacturing. In the literature, several classes of polymers have been shown to allow SMP behavior, including highly entangled amorphous polymers, crosslinked amorphous polymers (including castable SMPs), melt-miscible blends of semicrystalline and amorphous polymers, crosslinked semicrystalline polymers and their blends with rubber (shape memory rubber), and multiblock copolymers. The latter SMP class consists of phase-segregated linear block co-polymers having a hard segment and a soft segment. The hard segment is typically crystalline, with a defined melting point, and the soft segment is typically amorphous, with a defined glass transition temperature. In some embodiments, the hard segment is amorphous and has a glass transition temperature rather than a melting point. In other embodiments, the soft segment is crystalline and has a melting point or glass transition temperature. The melting point or glass transition temperature of the soft segment is substantially lower than the melting point or the glass transition temperature of the hard segment.

When the SMP is heated above the melting point or glass transition temperature of the hard segment or bulk material, the material can be shaped with complete relaxation of internal stress. This original shape can be memorized by cooling the SMP below the melting point or glass transition temperature of the hard segment or bulk material. When the shaped SMP is cooled below the melting point or glass transition temperature of the soft segment or bulk material while the shape is deformed, that temporary shape is fixed. The original shape is recovered by heating the material above the melting point or glass transition temperature of the soft segment or bulk material but below the melting point or glass transition temperature of the hard segment, in the case of phase-segregated linear block copolymers.

The shape memory effects are intimately linked to the polymer's structure and morphology and exist in many polymers with both thermoset and thermoplastic character. Examples of polymers used as SMPs include various polyethers, polyacrylates, polyamides, polysiloxanes, polyurethanes, polyether amides, polyurethane/ureas, polyether esters, and polyesters such as polycaprolactone.

Biomedical and other applications that require a predetermined response from a device based on a stimulus have attracted substantial attention in the literature. These so called “smart materials” include a class of polymers known as shape memory polymers (SMP). SMPs are materials that can be deformed and stored into a metastable shape indefinitely until activated by heat, light, or other stimuli. Upon activation SMPs recover a globally-stable shape. These materials' ability to controllably change in shape has been proposed as a method to improve numerous devices from arterial stents to suture anchors. Recent work has expanded the functionality of SMPs to include other properties such as biodegradability and the ability to elute drugs.

The inherent challenge associated with polymers that interact with the body is that this interaction is difficult to control post-implantation. The herein described invention will enhance the functionality of polymeric devices to include the ability to alter the properties of a coating by application of a stimulus. The properties that may be altered due to the stimuli-triggered mechanical deformation include, but are not limited to electrical properties, magnetic properties, optical properties, barrier properties, and mechanical properties. The ability to fundamentally change the polymer's reaction with the environment will be achieved through the activation of a shape change in a coated SMP. The coating will serve as a barrier layer limiting polymer-environment interaction and possibly providing other functionality such as electrical conductivity. The ability of the underlying polymer to undergo a triggered shape change will be utilized to cause strain-induced local or global failure in the barrier coating leading to increased interaction between the SMP and environment and possibly other changes in properties bestowed by the coating.

The development of biodegradable SMPs has not yet yielded an approved device due in part to the inherent challenges of developing a polymer system that can provide both long term mechanical stability and then degrade after the device is no longer needed. Triggered biodegradability of a device would address this concern, as addressed by the claimed invention.

### SUMMARY OF THE INVENTION

An embodiment of the claimed invention is directed to a shape memory polymer (SMP) that is manufactured in an elongated shape, compressed into a metastable shape, coated in a conformal coating, utilized in the metastable shape, then subjected to a mechanical stimulus, causing shape recovery and failure of the conformal coating, thereby allowing the polymer to be exposed to the environment.

A utility of the claimed invention is directed to its application in improved drug delivery mechanisms. Such a utility is achieved through the development of a sacrificial barrier coating that fails due to strains induced by memory effect.

It is an objective of the claimed invention to optimize the strain capacity of SMPs, characterize parylene coatings and determine their swelling profiles.

A further embodiment of the invention is directed to a shape memory polymer that is coated with a material, which upon exposure to a stimulus, causes the coating to weaken or fail at the site of exposure to the stimulus, thus revealing the shape memory polymer within.

### BRIEF DESCRIPTION OF THE DRAWINGS

FIG. 1 is a schematic that outlines a parylene deposition process which enables room temperature conformal coatings of biostable and bioinert barrier layers at specific thicknesses (e.g. from below 500 nm to more than of 10 microns).

FIG. 2 is a Dynamic Mechanical Analysis (DMA) graph which shows the glass transition temperature (Tg) as a drop in modulus of several degrees of magnitude.

FIG. 3 shows the measurement of tangent delta by DMA, where tangent delta is a ratio of the loss modulus (complex part) and the storage modulus (real part).

FIG. 4 is a Differential Scanning calorimetry (DSC) graph which shows the measurement of heat flow through samples in order to indicate relevant thermal events such as Tg.

FIG. 5 shows the swelling profiles of various copolymers of methyl acrylate (MA), methyl methacrylate (MMA), 2-hydroxyethyl methacrylate (2-HEMA) are determined after 1 day in phosphate buffered solution when uncoated, coated with parylene-C and coated with parylene N.

FIG. 6 shows the one-day swelling profiles of a parylene-C coating layer on acrylate copolymers.

FIG. 7 shows the one month swelling profiles of a parylene-C coating layer on acrylate copolymers.

FIG. 8 shows day and week swelling profiles for Parylene coatings that are strained to a variety of endpoints up to 40%.

### DESCRIPTION OF EXEMPLARY EMBODIMENTS

An embodiment of the invention is directed to a shape memory device, comprising a first material, able to memorize an original shape and being present in a deformed shape, and a second material, wherein the second material possesses at least one property that changes significantly upon recovery of the first material toward its memorized original shape, upon application of an external stimulus.

In certain embodiments, the shape memory devices of the present invention are sensitive towards an external stimulus, in particular mechanical forces, i.e. they show a shape memory effect after having been subjected to mechanical forces. The device comprises a first material which can memorize at least one shape, i.e. in accordance with the usual designation in the art the permanent shape. This material, in the device, is present in the deformed, i.e. temporary shape. However, in accordance with the present invention it is not required that the deformed state, i.e. the temporary shape (the designation as used again corresponds to the usual designation employed in the art for shape memory polymers) is fixed by means of interactions within the material (for example by chemical or physical interactions of soft or switching segments of shape memory polymers) since the device in accordance with the present invention comprises an additional second material fixing the deformed shape. In this respect the present invention in particular requires that the first material is elastic, in order to allow a recovery of the permanent shape after release of the fixation provided by the second material.

In other embodiments of the invention, the shape memory devices are sensitive to non-mechanical stimuli.

In certain embodiments, a shape memory polymer that is coated with a material is exposed to a stimulus, which causes the coating to weaken or fail at the site of exposure to the stimulus, thus revealing the shape memory polymer within.

Preferred first materials are accordingly elastomeric polymer networks, having a low glass transition temperatures (Tg) and being highly flexible. Further examples of preferred first materials are shape memory polymers with a switching temperature below the temperature at which the shape memory device in accordance with the present invention is to be used.

The device in accordance with the present invention accordingly fixes the deformed shape by an additional second material which may for example be provided in the form of a coating, partially or completely covering the article made from the first material. This second material displays a sufficient mechanical strength and physical integrity so that the temporary shape is secured. However, the second material is selected so that the application of a suitable external stimulus leads to a decrease of the mechanical strength of the second material or to the removal, partially or completely of the second material, so that the deformed shape cannot be maintained anymore. Instead the first material, no longer fixed by the second material, recovers its initial permanent shape, i.e. the deformed shape is lost and the permanent shape is formed.

As indicated above a suitable external stimulus in particular is a mechanical manipulation, such as a compression or a tensile stress. The use of a compression as external stimulus may in particular be of advantage for devices in accordance with the present invention which are manipulated before use with tools or using hands allowing the quick and easy application of a compressing force. This type of external stimulus may in particular be used with fragile or brittle materials and/or materials having a predetermined breaking point or the like which can be easily be deformed/destroyed by applying a compressing force. A further alternative is the application of a solvent in which selectively only the second material may be dissolved or at least swollen, so that the mechanical fixation is removed. Such a swelling leads to the formation of a gel phase of the second material so the mechanical integrity required for fixing the first material in the deformed shape is no longer given. When this second material however is subjected to a mechanical manipulation as mentioned above, the integrity and/or cohesion of the second material is disturbed so that the second material can no longer hold the first material in the deformed shape. In accordance with the present invention it is however not only envisaged to use second materials which are sensitive towards a compression or tensile load, i.e. mechanical forces, but also which loose, as outlined above, the required integrity and/or cohesion upon application of other stimuli, such as the solvent sensitive materials mentioned above, examples of which are polyethylene glycol and polyvinyl alcohol. Such solvent sensitive materials may in particular be of use for devices which shall detect the presence of such solvents in safety sensors etc. Other examples are medical devices which can be, immediately before use be placed into a container comprising the solvent towards which the materials is sensitive.

Examples of suitable second materials are materials which soften due to heating, so that the above described effect occurs after having subjected the device to a heat treatment. The softening according to the invention does also include liquidation, that may be followed by evaporation, and also sublimation. In a preferred embodiment of the invention the second material is in its solid or at least highly viscous state when fixing the first material and is liquid with a viscosity low enough to release the memory form of the first material. This phase or viscosity change of the second material can be achieved e.g. be heating, by mechanical or by chemical treatment, like e.g. shaking or treatment with ultrasonic waves that lower e.g. the viscosity, or exposure to chemicals like gases or liquids that reduce the melting point and/or viscosity of the second material.

In an embodiment of the invention, the second material is in its solid state, preferably in its crystalline or semicrystalline state, and in particular in its crystalline state, when fixed upon the first material, and is liquified to release the memorized form of the first material.

In certain embodiments of the invention, the second material may be a water based material, that preferably can be in the form of a gel or can be frozen and/or kept cool to fix the temporary form. The memorized form of the first material can then be released e.g. by heating to or above the melting point of the water based second material or by e.g. mechanical forces before it is unfreezed. Water based material means in the context of the present invention a composition, that includes at least about 20 wt %, preferably at least about 50 wt % and in particular at least about 85 wt % water. The water based material can include in dissolved or dispersed form ingredients like e.g. organic solvents, thickeners, gases, organic and inorganic substances. Using such ingredients alone or in any combination allows to finely adjust the response to the stimulus that weakens the fixation ability of the second material. In the weakened form, the water based material preferably is pure water, a solution, a dispersion or a gel.

In certain embodiments particularly for medical applications, the water based material can be a material that is compatible to the organism, in particular a physiological sodium chloride solution or the like, that may be sterilized and may contain further active agents and/or drugs.

A further option for the second materials are pH sensitive materials, i.e. materials which even disintegrate after having been subjected to a suitable change in pH value (in particular for devices which are used in liquid containing environments, e.g. in specific parts of the human or animal body like the acidic milieu of the stomach). Other examples are light sensitive second materials or materials which are susceptible towards a hydrolytic degradation or an enzymatic degradation. Examples of materials susceptible towards degradation are known to the skilled person and such materials in particular may be advantageously be used in medical devices, where disintegration may be facilitated/mediated by body fluids. In particular if the degradation products are not harmful such an embodiment is of high advantage for use in the medical field. In accordance with the present invention it is only necessary that the second material is able to fix and secure the deformed shape and that the second material is susceptible towards an external stimulus so that the first material, after the second material has been subjected to such a stimulus, recovers the remembered, i.e. permanent shape. Typical representatives of such suitable first materials in accordance with the present invention are thermoplastic polymers.

If a thermoplastic polymer is heated above a transitions temperature Tₜᵣₐₙₛ, that may be a glass transition temperature or a melting point, it becomes soft and principally capable of flowing. At this point or at a higher temperature it may loose its ability to fix the temporary form of a first material. The temperature increase may be induced directly by heating or by other energy sources like electromagnetic radiation that is absorbed, or by mechanical impact like rubbing or ultrasonic waves. For a more precise phase transition crystalline or semicrystalline thermoplastic polymers, that have a melting point, are preferred. The desired fixation power and transition temperature can be adjusted e.g. by the thickness of the second material including said thermoplastic polymer, the percentage it is in contact with the first material, its chemical composition and by blending of the thermoplastic polymer with one or several other polymers and/or with know polymer additives.

In the context of the present invention, thermoplastic polymers are preferably selected from the polymers or copolymers listed in the following, or include at least a monomer therefrom: vinyl polymers, polyethylene (PE), low density polyethylene (LDPE), high density polyethylene (HDPE), polypropylene (PP), styrene polymers, polystyrene (PS), styrene-acrylinitrile copolymer (SAN), styrene-butadiene-styrene copolymer (SBS), styrene-butadiene-crystallizable poly(.epsilon.-caprolactone) copolymer (SBC), styrene-crystallizable poly(.epsilon.-caprolactone) copolymer (SC), styrene-isoprene-styrene copolymer (SIS), styrene-ethylene-butylene-styrene copolymer (SEBS), acryinitrile-butadiene-styrene copolymer (ABS), butadiene-crystallizable poly(.epsilon.-caprolactone) copolymer (BC), poly(.epsilon.-caprolactone) (PCL), polycarbonate (PC), poly(tetramethylene carbonate), PC/ABS, poly(methyl methacrylate) (PMMA), polyacrylnitrile (PAN), polymethacrylnitrile (PMAN), polyvinylacetate (PVAc), polyvinylalkohol (PVA), polyvinylchloride (PVC), poly(vinylidene chloride) (PVDC), poly(vinylidene chloride) copolymer, polytetrafluorethylene (PTFE), polybutadiene, poly(dimethyl butadiene)polyoxymethylene (POM), polyester, poly(ethylene terephthalate) (PET), polydimethylsiloxane, polyamide (PA), cellulose ester, cellulose acetate, cellulose acetate propionate, cellulose acetate butyrate, cellulose propionate, cellulose triacetate, polyurethanes, poly(ether esters), poly(ether amides), polyether, poly(phenylene oxide) (PPO), polypropylene oxide), PPO/PS, poly(butylene terephthalate) (PBT), polysulfone (PSU), aromatic polyester (APE), polyamideimide (PAI), poly(ether imide) (PEI), poly(ether sulphone) (PES), poly(ether ether ketone) (PEEK), poly(phenylene sulfide) (PPS), ethylene-propylene-diene copolymer (EPDM), EPDM/PP, natural rubber-PP, polyethylene-vinyl acetate (EVA), EVA/PVDC, nitrile rubber/PP, and acrylate polymers such as methyl acrylate (MA), methyl methacrylate (MMA), 2-hydroxyethyl methacrylate (2-HEMA), 2-2 dimethoxy-2-phenylacetophenone (DMPA) and bisphenol A ethoxylate diacrylate (BPAEDA) or modifications or derivatives thereof.

The thermoplastic polymer can be selected by known physical and chemical data of the polymers and by usual experiments to best fit for the given application in terms of e.g. transition temperature; mechanical strength for fixation; processability; compatibility with the first material, e.g. that it can depending on the desired application be removed easily, or to the contrary that the adhesion is strong enough to remain on the first material for a second programming step; and/or compatibility with the surrounding in the given application, in particular biocompatibility and non-toxicity.

The first material to be employed in accordance with the present invention may be any material which is able to maintain at least one shape in memory, i.e. which is able to recover the original shape after a deformation (and the fixation of the deformed shape by means of the second material). Suitable examples thereof are shape memory polymers as for example illustrated in the prior art references mentioned above. However, as outlined above, any material which is able to remember one shape may be employed in accordance with the present invention. Accordingly the present invention also contemplates to use as first material, as already indicated above, elastic materials, in particular rubber materials of natural or synthetic origin. Also such elastic materials, such as natural or synthetic rubber, including EPDM materials and the like, are materials which, after an elastic deformation, display the ability to return to the non-deformed state after the external force (i.e. in the present invention the restraining coating of the second material) fixing the deformed shape is removed. Elastic materials according to the invention also include resilient, springy and superelastic materials and also include such metallic materials, alloys, composites, and even complex devices. Preferred in this respect are rubber materials in the form of polymer networks having main chain segments providing a domain having a rather low glass transition temperature.

One further possibility in this respect is the use of a shape memory polymer which has been programmed and accordingly provides one permanent shape and at least one temporary shape (depending on the number of switching segments) so that in addition to the permanent shape and the temporary shape as enabled by the shape memory polymer a further deformed shape is made possible. In this embodiment the temporary shape as programmed onto the shape memory polymer is deformed further and this additional deformed shape is then fixed in accordance with the present invention using the second material. Accordingly in such an embodiment the first shape change occurs when the second material is no longer able to secure the deformed shape—the shape memory polymer returns to the temporary shape from which the permanent shape may be recovered upon initiating the shape memory effect of the shape memory polymer.

By this embodiment it is easily and cost-efficiently possible to obtain a device with two shapes in memory, the permanent and the temporary shape, and further on the deformed shape (a so called triple-shape material). The same effect could be achieved with a device according to the invention including a first material with a permanent form that is deformed and fixed with a second material into a deformed shape and which is then deformed again and fixed with a different second material.

In addition it would be possible to again deform the deformed shape of a triple-shape material and fix this with a different second material to get a quad-shape material, and so on. Alternatively a triple-shape shape memory polymer with a permanent and two programmed temporary forms could be deformed and fixed accordingly. In this way very complex and flexible programming steps can be performed.

Depending on the intended use, the stimuli to switch the temporary to the permanent form and to release a deformed form, can be different or the same. If more than one deformed form is present, also the release of each deformed form can be affected by the same or different stimuli. In a preferred embodiment of the present invention at least one of said stimuli can be a predefined temperature and in particular all stimuli are differently predefined temperatures.

However, the present invention is concerned with the fixation of a deformed state of a first material, which is able to memorize the original shape, using the second material, so that the device is able to recover this original shape when the second material no longer secures the deformed shape.

In an embodiment of the invention, the second material partially or completely covers or at least fixes the article formed from the first material in the deformed shape. The type of coating, such as coating pattern, coating thickness etc. depends from the desired end use and the type of the first materials as well as the type of article and the degree of deformation. Complete coatings might in particular be required when the first material displays a strong tendency to recovering the original shape, while partial coatings may be suitable in particular in fields of application where the second material is rather expensive so that only the minimum required amount is to be used. However, these illustrative explanations shall not be construed as limitation since the skilled person will be in a position to determine the appropriate type of coating for the desired use and selected composition. A further alternative is the use of fibers or bands of the second material as well as sheets thereof which are used to be wrapped around the article formed from the first material being in the deformed shape. Thereby the deformed shape may be fixed as well without providing a coating of the second material onto the article formed from the first material.

As outlined above the second material may be selected from suitable materials providing a desired sensitivity towards an external stimulus. The following options are in particular envisaged by the present invention: thermo-sensitive materials application of heat softens the second material so that first material returns to permanent shape, i.e. non-deformed shape; light-sensitive materials application of light softens or degrades the second material so that first material returns to permanent shape, i.e. non-deformed shape; solvent-sensitive materials application of solvent selectively softens or removes the second material so that first material returns to permanent shape, i.e. non-deformed shape; pH-sensitive materials variation of pH-value softens, degrades or removes the second material so that first material returns to permanent shape, i.e. non-deformed shape; and materials sensitive towards a magnetic field application of magnetic field softens the second material so that first material returns to permanent shape, i.e. non-deformed shape

The second material furthermore may be selected to have suitable properties, such as biocompatible materials, erodible materials, materials which degrade, for example by hydrolytic or enzymatic processes, crystalline, semicrystalline or amorphous materials and the like, depending in particular from the desired end use of the device formed.

As already mentioned above, it is preferred that the second material is biocompatible, what is particularly relevant for materials, with which humans, animals, or a sensitive environment can get in contact. Biocompatibility is exceptionally relevant for medical devices that are developed to be implanted into humans or animals, and the materials used for such devices should pass the mandatory biocompatibility and toxicity tests. The requirements concerning biocompatibility also hold for the first material, if this can get in contact with humans, animals, or a sensitive environment.

Erodable materials according to the invention include materials that are ablated or get brittle or fragile on continued exposure to an external stimulus. The external stimulus can be e.g. ambient air, exhaust fumes, specific gases, light, in particular UV light, high energy radiation like e.g. X-rays, alpha, beta or gamma rays, heat, smoke, water, e.g. waste water, solvents, microbes, et cetera. The external stimulus can also be a mechanical impact like fine particles in the passing gas, air or liquid, rubbing contact to a surface, et cetera. In this embodiment the second material is selected from materials that are known to be erodible in the aforementioned sense for a given external stimulus. In a predefined time of exposure to said external stimulus, that is effective integrally, the second material will be eroded to such an extent, that the first material is released and changes back to its permanent form. The time for this erosion varies with the surface, the mass and the chemical composition of the used material and can easily be adjusted by usual experiments. A device of the invention with such a second material can advantageously be used as a sensor or an actor (e.g. switch, valve), that detects a predefined integral magnitude (e.g. amount or dose) of an external stimulus and indicates this by a change in shape of the first material, wherein this change in shape can also be used to actuate a mechanical, electromechanical, optomechanical, etc., device like a switch or a valve.

Such sensors and actuators of the invention can particularly advantageously be used as fire and smoke sensors as mentioned above, for environmental surveillance and protection of human beings and equipment. Examples for such uses are the detection of i) water pollution, ii) the corrosiveness of ambient air or e.g. cooling liquids, iii) an amount of UV or high energy radiation, gas or chemicals that is harmful for an organism like a human being, an animal or a plant, or for a technical equipment, iv) a microbic affection, v) unwanted products in a reaction mixture, gas or solvent, and vi) the amount of fine particles in the ambient air. The erosion by e.g. an equipment that is rubbing on the device of the invention, can e.g. be used to detect an excessive vibration of this equipment or to indicate its maximum reliability, if it's only rubbing to the device of the invention when being operative under load.

A special group of the aforementioned erodible materials are materials which degrade, for example by hydrolytic or enzymatic processes. As described above, devices including such materials as second material can be used to detect the existence of such an environment qualitatively as well as quantitatively. A further advantage of this embodiment of the device of the invention is particularly relevant for medical devices, that are designed to be implanted into the human or animal body, as it allows that the second material is eroded after implantation. This erosion can be used to affect the release of the first material, but can also be used to only remove the second material from the place of implantation, if the second material has already lost its fixing-ability under the impact of a different stimulus.

The shape memory device in accordance with the present invention may be prepared in a conventional manner using the forming techniques described in the art for shape memory polymers. The first material has to be provided in the original i.e. permanent shape for the desired article of manufacture. Subsequently the article is deformed until the desired deformed shape is obtained, which in turn then is fixed using the second material, for example by applying a partial or complete coating using conventional techniques. The article obtained accordingly is fixed in the deformed shape and the original shape can only be recovered by applying an external stimulus as indicated above.

The shape memory devices in accordance with the present invention may in particular be used in applications where a change in shape in response to an external mechanical force is suitable, for example in pressure sensors. Other applications are areas where a shape change in response to a tensile force or compression force is desired, for example sensors, but also medical devices as well as other articles of manufacture, such as toys etc. Further fields of application are medical devices, such as in particular stents, which may be fixed in a compressed, i.e. small diameter shape, by the second material, which in turn then is softened or removed after insertion so that the stent recovers its original shape.

The invention herein describes the coating of a shape memory polymer or polymer coated shape memory alloy device with a barrier coating. This invention relates to the coating of the device and the subsequent mechanical deformation of this coating to control polymer-environment interactions and the properties of the coating. The preferred embodiment of this invention consists of coating a shape memory polymer that has been compressed and fixed using the shape memory effect into a metastable shape.

An embodiment of the invention is directed to a shape memory device, comprising a first material, able to memorize an original shape and being present in a deformed shape, and a second material, wherein the second material possesses an ability to fix the first material in the deformed shape, and loses its ability to fix the first material in the deformed shape upon application of an external stimulus.

In an embodiment of the claimed invention, the coating consist consists of poly(para xylylene), or one of its derivatives, that can be applied through room temperature chemical vapor deposition polymerization. The thickness of the coating is not limited by this invention except in that it must be able to serve as a semi-permeable or impermeable barrier to the environment prior to activation. After the activation of the shape memory effect the coating no longer serves as an effective barrier between the polymer and the environment. This triggered change is associated with the mechanical deformation (shape change in this case of the SMP) which strains the coating to the point of failure allowing for the underlying polymer to interact with the environment.

Another embodiment of the invention is characterized by the coating of a SMP while strained in tension or in a globally-stable state. The polymer could, after coating, be heated and further deformed beyond the strain capacity of the coating and then utilized. This would readily allow polymer-environment interaction until the activation of the SMP upon which the coating would regain its integrity and begin to act as an impermeable coating or semi-permeable membrane, perhaps trapping a drug or other substance inside the coating.

A further embodiment of the invention is characterized by the coating of an SMP that is fixed in a metastable state of some complex deformation. The device upon activation would recover tensile strain in some areas thus reinforcing the coating and strain the coating in other areas where compressive strain is recovered.

In an embodiment of the invention, the polymer-environment interaction could include, but would not be limited to the elution of a chemical substance and biodegradation.

In a further embodiment of the invention, the activation of the SMP would be both activation and SMP system dependent. Options for this activation include, but are not limited to laser-activation, changes in pH or ion content of the environment, irradiation with infrared radiation, direct application of heat or heat generated through magnetic induction.

Another aspect of the claimed invention is the matching of the strain-to-failure of the polymer coating with the deformation that will be recovered upon activation of the device. In one embodiment this could include the use of parylene-c, parylene-d, parylene-n or any of the other derivatives of poly(para xylylene) or copolymers of these derivatives. In other embodiments the coating can be of any polymer, metal, or ceramic that can be applied as a thin film and fulfills the functions described herein.

Another embodiment of the claimed invention utilizes the shape memory effect associated with shape memory alloys or ceramics to trigger the deformation that causes the triggered change in the polymer-environment interaction.

An alternate embodiment of the invention is directed to the use of a shape memory polymer for drug delivery. In such an embodiment, a drug or other compound or molecule is enclosed within a shape memory polymer or within a vessel enclosed by the shape memory polymer such that the triggered shape memory effect mechanically deforms an external coating exposing the environment to this contained material.

### WORKING EXAMPLES

### Materials and Methods

Methyl acrylate, 2-hydroxyethyl methacrylate, methyl methacrylate, bisphenol a ethoxylate diacrylate (BPAEDA) (Mw ˜512 g/mol), phosphate buffered saline solution (PBS), and 2,2-dimethoxy-2-phenyl acetophenone (DMPA) were purchased from Sigma Aldrich (St Louis, Mo.). Dichloro[2.2]paracyclophane (precursor for Parylene-C) and [2.2]paracylcophane (precursor for Parylene-N) were purchased from SCS Coatings (Indianapolis, Ind.). All chemicals were used as received without further purification.

### Synthesis of Polymer Networks

Acrylate networks were synthesized by free radical polymerization using 0.5 wt % DMPA as a photoinitiator and 5 wt % BPAEDA as a crosslinking Mixtures of the (meth)acrylate monomers, the BPAEDA crosslinking monomer, and the photoinitiator were injected between two glass slides with a separated by a glass spacer 1.2 mm thick or in a cylindrical mold depending on the subsequent testing. Polymerization was performed using a Translinker crosslinking chamber with five overhead 365 nm UV bulbs (Cole-Parmer) for 120 minutes. A thermal post-cure was performed at 90° C. for 1 hour to help ensure complete polymerization.

### Compression of Shape Memory Polymer Devices

All polymer devices that were compressed were compressed after post-curing and before coating. Pre-compressed samples were cylindrical of dimensions approximately 10 mm in length 12 mm in diameter. Samples were strained to various endpoints in compression at the temperature of the peak of tangent delta (at 1 Hz) as determined by dynamic mechanical analysis. Devices were compressed at 1 mm/min.

### Coating of Shape Memory Polymer Devices

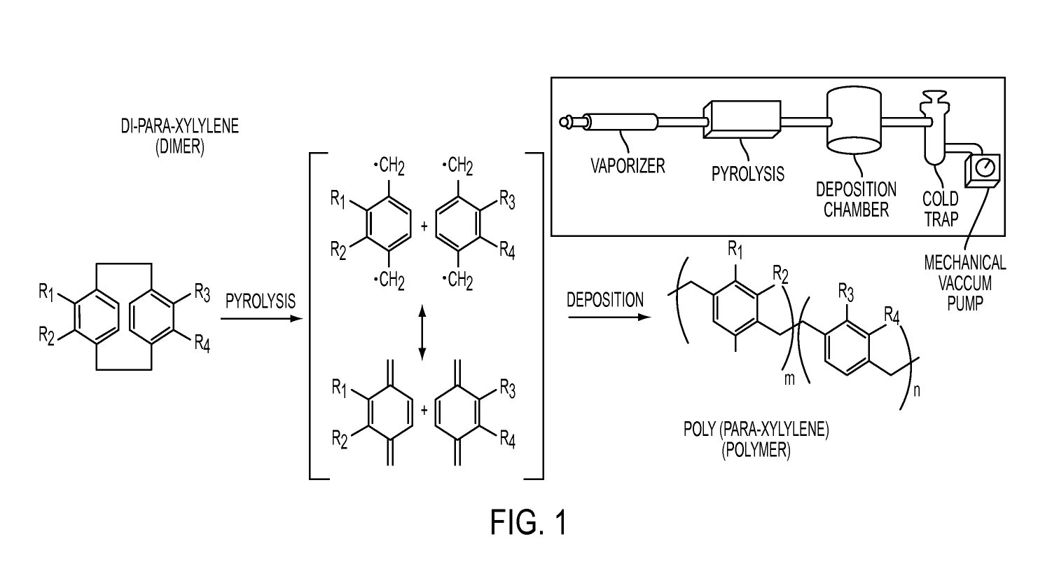

Shape memory polymer devices were coated in Parlyene-C or Parylene-N by a chemical vapor deposition polymerization process in a tumbling apparatus rotating at 10 rpm. The thickness of the coating was controlled by changing the amount of the Parylene-N or Parylene-C precursor and was confirmed by ellipsometry on flat silicon wafers. For Parylene-C depositions, dichloro[2.2]paracyclophane was vaporized at 175° C. and pyrolized at 680° C. deposition and polymerization followed at room temperature at a pressure of approximately 15 mTorr. For Parylene-N depositions, [2.2]paracyclophane was vaporized at 160° C. and pyrolized at 650° C. deposition and polymerization followed at room temperature at a pressure of approximately 25 mTorr Coating of the shape memory polymer devices was carried out under identical conditions for both compressed and unstrained polymer devices (FIG. 1).

### Recovery of Coated and Compressed Shape Memory Polymer Devices

Recovery of compressed shape memory polymer devices was carried out by placing the compressed devices in an oven set for the peak of tangent delta (1 Hz) as determined by dynamic mechanical analysis. Devices were allowed to recover for 30 minutes and subsequently measured. All devices showed approximated 100% recovery.

Differential Scanning calorimetry

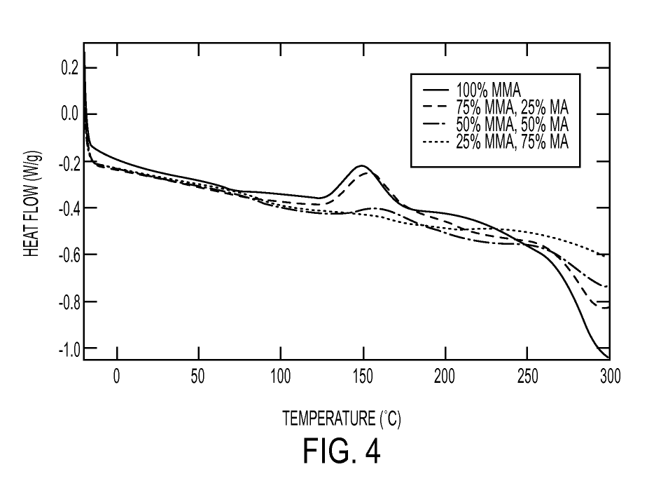

DSC was performed on uncompressed and uncoated shape memory polymers on a Mettler Toledo DSC 1 (Columbus, Ohio). Samples were heated from room temperature to 150° C., cooled to −20° C., and subsequently heated to 300° C. Data shown is of only the second heating ramp. All heating and cooling rates were fixed at 10° C./min. Tests were conducted in a nitrogen atmosphere (FIG. 4).

### Dynamic Mechanical Analysis

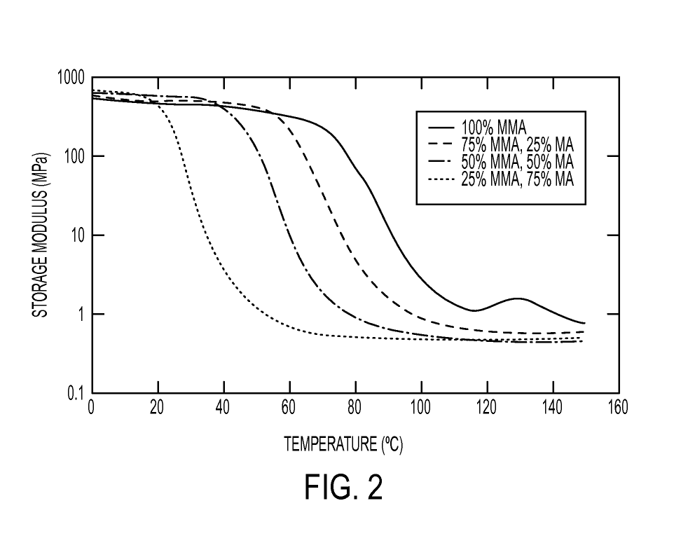

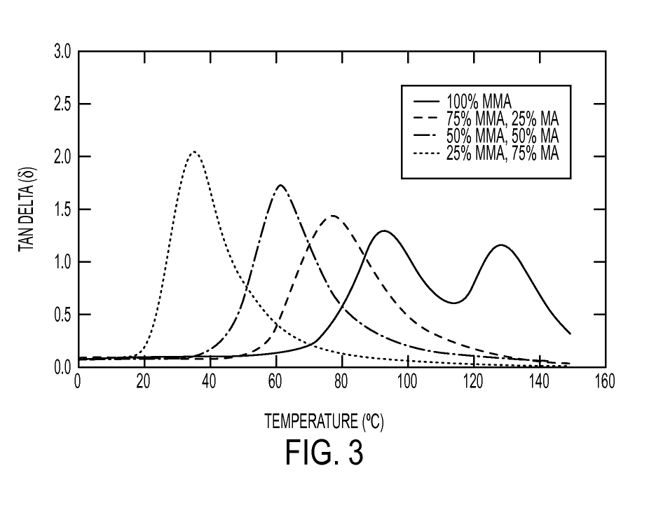

DMA was performed on uncompressed and uncoated shape memory polymers on a Mettler Toledo DMA 861e/SDTA (Columbus, Ohio). Samples were cut into discs approximately 1.2 mm thick and 3 mm in diameter. The mode of deformation was shear and strain was limited to a maximum of 0.25%. Samples were tested between 0-150° C. at a heating rate of 2° C./min. Tests were conducted in a nitrogen atmosphere (FIGS. 2 and 3).

### Swelling Tests

Shape memory polymer cylinders approximately 10 mm in length and 12 mm in diameter were subjected to phosphate buffered saline solution for various time points between 1 day and 1 month at room temperature. Devices that had been coated and compressed and coated were also tested under the same conditions. Swelling was determined by briefly removing the sample from PBS, removing excess PBS from the surface, and recording the mass of the polymer device. This mass was compared to the initial mass and swelling calculated by equation

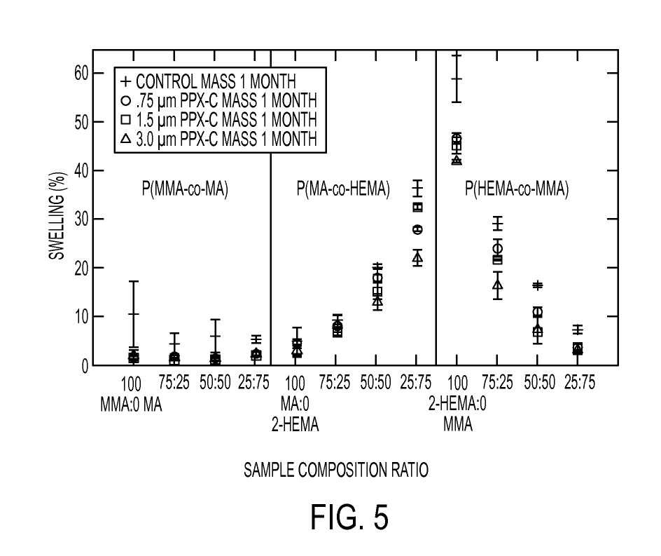

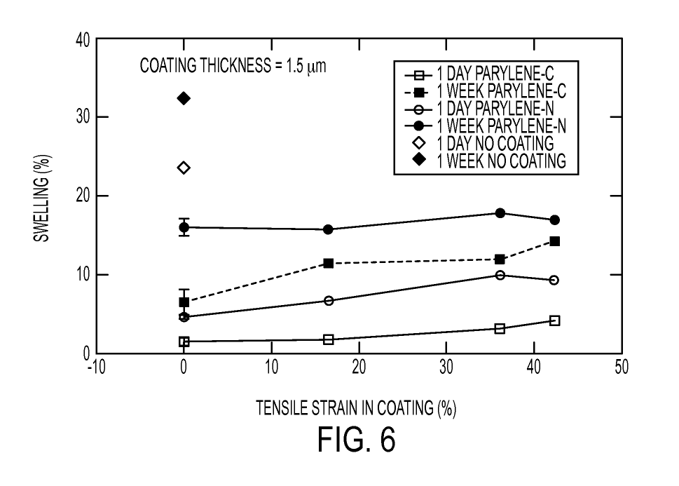

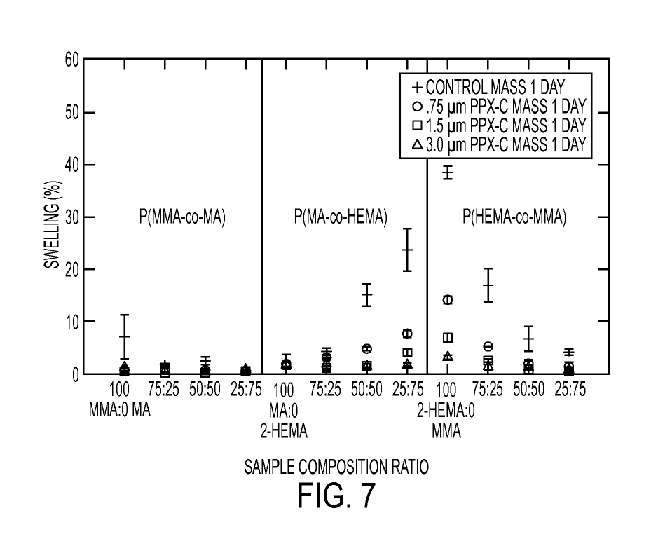

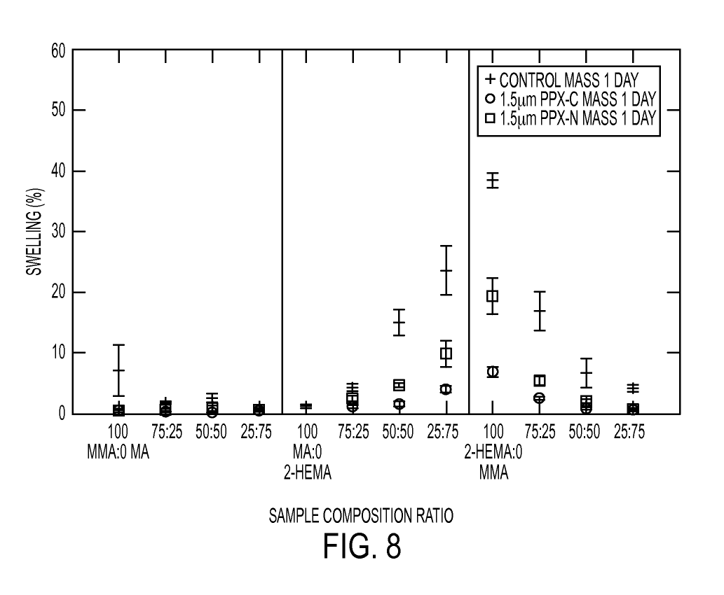

1. All swelling tests were performed in triplicate (FIGS. 5-8).

$$Swelling ( \% ) = \frac{Swollen Mass - Initial Mass}{Initial Mass} \qquad Equation 1$$

### Drug Elution Method

An embodiment of this invention concerns the post-implantation triggered release of a drug from a shape memory polymer device. The device is manufactured and functions as below:

A mixture of monofunctional acrylate monomers 50 wt % methyl acrylate and 50 wt % 2-hydroxyethyl methacrylate can be polymerized by free radical polymerization with poly(ethylene glycol)diacrylate serving as a crosslinker and in the presence of lyophilized vancomycin, a common antibiotic. This reaction encapsulates antibiotic in the polymer matrix. The device can be manufactured in the form of a cylinder. This drug-loaded cylinder is then be compressed to 66% of its original size by heating the polymer to a temperature in the middle of its glass transition and applying the necessary force. The compressed cylinder is fixed in this state by cooling to a temperature below its glass transition while maintaining the compression. The compressed device is coated with 3 μm of Parylene-C. This device could then be implanted subcutaneously near the site of a possible infection. The coating on the device effectively prevents elution of the drug or swelling of the polymer device with fluid. At a time in the future post-implantation a physician could decide to begin treating with the antibiotic. At this time an external heat source such as a focused infrared light source would be applied to the implanted device causing an expansion of the device. This will cause significant damage to the applied Parylene-C coating, and will permit swelling of the polymer device with water and subsequent elution of the vancomycin.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A shape memory device, comprising a first material, able to memorize an original shape and being present in a deformed shape, and a second material, wherein the second material possesses at least one property that changes significantly upon recovery of the first material toward its memorized original shape upon application of an external stimulus.

2. The shape memory device of claim 1, wherein the external stimulus is mechanical.

3. The shape memory device of claim 1, wherein the external stimulus is a change in temperature, pressure, pH, electric field, or magnetic field.

4. The shape memory device of claim 1, wherein the second material partially or completely covers the first material.

5. A shape memory device, comprising a first material, able to memorize an original shape and being present in a deformed shape, and a second material, wherein the second material possesses an ability to fix the first material in the deformed shape, and loses its ability to fix the first material in the deformed shape upon application of an external stimulus.

6. The shape memory device of claim 5, wherein the second material upon application of an organic solvent or other chemical stimulus has a physical state selected from the group consisting of brittleness, loss of integrity and loss of cohesion.

7. The shape memory device of claim 1, wherein the second material is susceptible to degradation by a means selected from the group consisting of hydrolytic and enzymatic means.

8. The shape memory device of claim 5, wherein the external stimulus is mechanical.

9. The shape memory device of claim 5, wherein the external stimulus is a change in temperature, pressure, pH, electric field, or magnetic field.

10. The shape memory device of claim 5, wherein the second material partially or completely covers the first material.

11. The shape memory device of claim 1, wherein the second material is a thermoplastic polymer.

12. The shape memory device of claim 5, wherein the second material is a thermoplastic polymer.

13. The shape memory device of claim 1 wherein the device is selected from the group consisting of sensors, medical devices, stents, microelectronics, flexible electronics, solvent detectors, valves, flaps, fire sensors, smoke sensors, devices that react to fire, devices that react to radioactivity, dosimeters, and devices that react to a predefined integral magnitude of the external stimulus.

14. The shape memory device of claim 5 wherein the device is selected from the group consisting of sensors, medical devices, stents, microelectronics, flexible electronics, solvent detectors, valves, flaps, fire sensors, smoke sensors, devices that react to fire, devices that react to radioactivity, dosimeters, and devices that react to a predefined integral magnitude of the external stimulus.

15. The shape memory device of claim 5, wherein the second material is a light sensitive shape memory material.

16. The shape memory device of claim 1, wherein the external stimulus is a solvent.

17. The shape memory device of claim 5, wherein the external stimulus is a solvent.

18. The shape memory device of claim 1, wherein the external stimulus is light.

19. The shape memory device of claim 5, wherein the external stimulus is light.

20. The shape memory device of claim 5, wherein the external stimulus is selected from the group consisting of heat sufficient to flow or heat sufficient to move through a thermal transition such as a melt, sublimation, evaporation, dissociation, decomposition, crystallization.

21. The shape memory device of claim 1, wherein the external stimulus is selected from the group consisting of heat sufficient to move through a thermal transition such as a melt, dissociation or glass transition.

22. The shape memory device of claim 1, wherein the external stimulus is an ultrasonic frequency.

23. The shape memory device of claim 5, wherein the external stimulus is an ultrasonic frequency.

24. The shape memory device of claim 5, wherein the external stimulus is selected from the group consisting of exposure to chemicals that decrease the melting point or Tg and exposure to chemicals that decrease the modulus of the second material.

25. The shape memory device of claim 5, wherein the external stimulus is selected from the group consisting of causing ablation, increasing brittleness and increasing fragility of the second material.

26. The shape memory device of claim 5, wherein the external stimulus is selected from the group consisting of ambient air, exhaust fume, gas, light, UV light, high energy radiation, heat, smoke, water, waste water, solvent, microbe, mechanical impact, polluted water, corrosive ambient air, corrosive liquid, harmful chemical, reaction product in chemical synthesis, fine particle, and vibration.

27. The shape memory device of claim 1, wherein the external stimulus is selected from the group consisting of ambient air, exhaust fume, gas, light, UV light, high energy radiation, heat, smoke, water, waste water, solvent, microbe, mechanical impact, polluted water, corrosive ambient air, corrosive liquid, harmful chemical, reaction product in chemical synthesis, fine particle, and vibration.

28. The shape memory device of claim 1, wherein the first material, second material, or both materials is selected from the group consisting of a material in a crystalline state, semicrystalline state, water based material, pH sensitive material, light sensitive material, material with a non-harmful degradation product, biocompatible material, material sensitive to a magnetic field, foam, and erodible material.

29. The shape memory device of claim 5, wherein the first material, second material, or both materials is selected from the group consisting of a material in a crystalline state, semicrystalline state, water based material, pH sensitive material, light sensitive material, material with a non-harmful degradation product, biocompatible material, material sensitive to a magnetic field, foam, and erodible material.

30. The shape memory device of claim 1 wherein the first material is selected from the group consisting of thermoset shape memory polymer, thermoset shape memory polymer foam, thermoplastic shape memory polymer, thermoplastic shape memory polymer foam, shape memory alloys or composites wherein at least one component is of this group.

31. The shape memory device of claim 5 wherein the first material is selected from the group consisting of thermoset shape memory polymer, thermoset shape memory polymer foam, thermoplastic shape memory polymer, thermoplastic shape memory polymer foam, shape memory alloys or composites wherein at least one component is of this group.

32. A method of using the shape memory device of claim 1, comprising the steps of: a) inserting the shape memory device into a human body, b) placing the shape memory device in a desired location within the human body, c) applying an external stimulus, and d) expanding the shape memory device into the original shape.

33. A method of using the shape memory device of claim 5, comprising the steps of: a) inserting the shape memory device into a human body, b) placing the shape memory device in a desired location within the human body, c) applying an external stimulus, and d) expanding the shape memory device into the original shape.

34. A method for facilitating the interaction between a shape memory device and its environment, wherein the shape memory article comprises a shape memory material, comprising the steps of:
deforming and fixing the shape memory article into a metastable state;
coating the shape memory article with a coating or partial coating;
triggering a mechanical deformation of the shape memory device;
causing the coating to be strained to a point where a property of the coating is altered; and
facilitating the interaction between the underlying shape memory device and its environment.

35. A method for facilitating the interaction between a shape memory device and its environment, wherein the shape memory article comprises a shape memory material, comprising the steps of:
deforming and fixing the shape memory article into a metastable state;
coating the shape memory article with a coating or partial coating;
triggering a property change in the coating;
enabling the shape memory device to recover towards its memorized shape; and
facilitating the interaction between the underlying shape memory device and its environment.

36. The method of claim 35 wherein the property of the coating that is altered is the permeability, electrical conductivity, piezoelectric properties, hydrophobicity, optical transparency.
